(19) United States
(12) Patent Application Publication
(10) Pub. No.: US 2026/0041716 A1
(43) Pub. Date: Feb. 12, 2026

(54) PHARMACEUTICAL COMPOSITION FOR TREATING WOUND REPAIR AND PREPARATION METHOD THEREOF

(71) Applicant: Gwo Xi Stem Cell Applied Technology Co., Ltd., Hsinchu County (TW)

(72) Inventors: Ruei-Yue Liang, Hsinchu County (TW); Kai-Ling Zhang, Hsinchu County (TW); Po-Cheng Lin, Hsinchu County (TW); Chia-Hsin Lee, Hsinchu County (TW); Ming-Hsi Chuang, Hsinchu County (TW); Yu-Chen Tsai, Hsinchu County (TW); Chu-Lin Tsai, Hsinchu County (TW)

(21) Appl. No.: 18/918,126

(22) Filed: Oct. 17, 2024

(30) Foreign Application Priority Data
Aug. 8, 2024 (TW) 113129821

(51) Int. Cl. A61K 35/28; A61K 38/18; A61K 38/19; A61K 38/28; A61K 38/48; C12N 5/0775

(52) U.S. Cl. CPC A61K 35/28; A61K 38/1866; A61K 38/1891; A61K 38/193; A61K 38/28; A61K 38/4886; C12N 5/0668; C12N 2500/24; C12N 2500/34; C12N 2500/36; C12N 2500/38; C12N 2500/44; C12N 2500/46; C12N 2500/99; C12N 2501/12; C12N 2501/16; C12N 2501/335; C12N 2501/39; C12N 2501/395; C12N 2501/71; C12N 2501/998

(57) ABSTRACT

The present invention can provide a pharmaceutical composition for treating wound repair and a preparation method thereof. The pharmaceutical composition at least includes an extracellular vesicle, insulin, and a plurality of growth factors, wherein the concentration of the extracellular vesicle in the pharmaceutical composition is (57.53±3.79)×10⁸ particles/mL; the concentration of the insulin in the pharmaceutical composition is 34,870.83±7,335.13 mU/L; these growth factors are ANGPTL4, HGF, G-CSF, PDGF-AA, VEGF-A, IL-18BPa, COMP, MMP-1, MMP-2, MMP-3, MMP-7, MMP-9, MMP-10, and MMP-12; and the extracellular vesicle contains a CLU protein, a TIMP1 protein and a YWHAB protein.

### CROSS-REFERENCE TO RELATED APPLICATION

The present application claims priority from Taiwanese Patent Application No. 113129821 filed on Aug. 8, 2024, the contents of which are incorporated herein by reference in their entirety.

### FIELD OF TECHNOLOGY

The present invention relates to a pharmaceutical composition and a preparation method thereof, and in particular to a pharmaceutical composition obtained from a conditioned medium obtained after cell culture and a preparation method thereof.

### BACKGROUND

In recent years, it has been found that a conditioned medium (CM) of mesenchymal stem cells (MSCs) cultured in a serum-free medium has the potential to promote body repair, health care and disease treatment. Disease treatment indications include cardiovascular diseases, traumatic diseases, lung diseases, nervous system diseases, immune diseases, liver diseases, endocrine diseases, skin diseases, gastrointestinal diseases, kidney diseases, hematologic diseases, cancer tumor diseases, gynecological diseases, mental diseases, urinary system diseases, ophthalmic diseases, dental diseases, etc., which have a wide range of medical industry utilization values.

It has been found according to research that, the ingredients in the conditioned medium after MSC culture contain growth factors or extracellular vesicles (EVs), and these ingredients can be used for the treatment and application of the aforementioned indications. Therefore, in the prior art, the secretion of the EVs or the growth factors is improved through physical stimulation or external pressure given on cells. However, these technologies can only increase the secretion of the EVs or the growth factors and cannot increase the effective ingredients for treating specific diseases.

### SUMMARY

Therefore, the applicant provides a pharmaceutical composition derived from a conditioned medium and a preparation method thereof. The pharmaceutical composition has extracellular vesicles, insulin, and specific types and ratios of growth factors, and can be used for the treatment of wound healing.

Specifically, the present invention can provide a method for preparing a pharmaceutical composition for treating wound repair, which includes: (a) a step of preparing a pharmaceutically effective mother liquor: implanting a starting stem cell into a basal medium, culturing the starting stem cell under a condition of an ambient temperature of 35.5-39.5° C. and a CO₂ concentration of 5% for more than at least 2 days, and then removing the starting stem cell to obtain a pharmaceutically effective mother liquor suitable for wound repair; and (b) a step of preparing a pharmaceutical composition: treating the pharmaceutically effective mother liquor by a purification and refining means to obtain a pharmaceutical composition suitable for treating wound repair; wherein the starting stem cell is a human mesenchymal stem cell, or a cultured stem cell obtained by culturing the human mesenchymal stem cell; the pharmaceutically effective mother liquor at least contains extracellular vesicles, insulin, and a plurality of growth factors as pharmaceutically effective ingredients; and the basal medium is a serum-free medium composed of a growth factor or growth hormone, a redox substance, an energy metabolism-related ingredient, and other nutrient sources.

According to a specific embodiment of the present invention, the growth factor or growth hormone of the basal medium is at least one selected from the group consisting of activin-A, corticosterone, exendin-4, a hepatocyte growth factor (HGF), pentagastrin, progesterone, retinol, and triiodol-1-thyronine.

According to a specific embodiment of the present invention, the redox substance of the basal medium is at least one selected from the group consisting of catalase, glutathione, and superoxidase dismutase.

According to a specific embodiment of the present invention, the energy metabolism-related ingredient of the basal medium is at least one selected from the group consisting of D(+)-galactose, ethanolamine, L-carnitine, linoleic acid, linolenic acid, lipoic acid, nicotinamide, putrescine, and sodium selenite.

According to a specific embodiment of the present invention, the other nutrient sources of the basal medium are at least one selected from the group consisting of albumin, D,L-alpha-Tocopherol, D,L-alpha-Tocopherol acetate, holo transferrin, and retinol acetate.

According to a specific embodiment of the present invention, the cultured stem cell is obtained by implanting the human mesenchymal stem cell into an auxiliary medium and culturing for at least 24 hours; the auxiliary medium is a keratinocyte serum-free medium containing fetal bovine serum, N-acetyl-L-cysteine, and ascorbyl phosphate.

According to a specific embodiment of the present invention, the cultured stem cell is obtained by implanting the human mesenchymal stem cell at a cell density of 6,000 to 15,000 cells/cm² into an auxiliary medium for at least 24 hours; and the auxiliary medium is a keratinocyte serum-free medium containing fetal bovine serum, N-acetyl-L-cysteine, and ascorbyl phosphate.

According to a specific embodiment of the present invention, the purification and refining means include: centrifuging the pharmaceutically effective mother liquor at an acceleration of 300 g for 5 minutes, then removing a precipitate, and then centrifuging at an acceleration of 4,000 g for 20 minutes, and removing a precipitate again.

According to a specific embodiment of the present invention, the purification and refining means further include: filtering a stem cell release solution after centrifugation by a filter with a pore size of 0.22 μm.

A pharmaceutical composition for treating wound repair is provided, which is prepared by the above-mentioned preparation method, and the pharmaceutical composition at least includes an extracellular vesicle, insulin, and a plurality of growth factors, wherein the concentration of the extracellular vesicle in the pharmaceutical composition is (57.53±3.79)×10⁸ particles/mL; the concentration of the insulin in the pharmaceutical composition is 34,870.83±7,335.13 mU/L; these growth factors are ANGPTL4, HGF, G-CSF, PDGF-AA, VEGF-A, IL-18BPa, COMP, MMP-1, MMP-2, MMP-3, MMP-7, MMP-9, MMP-10, and MMP-12; and the extracellular vesicle contains a CLU protein, a TIMP1 protein and a YWHAB protein.

According to a specific embodiment of the present invention, in addition to being used for wound repair-related treatments such as repair of diabetic wounds, repair of pressure sores, corneal repair, repair of xerophthalmia, repair of various difficult wounds or wounds that have not healed for a long time, skin repair or maintenance in medical cosmetology, the pharmaceutical composition can also be used for angiogenesis, immune diseases, heart protection, treatment of cardiovascular diseases, heart damage repair, treatment of ischemic heart diseases, neurogenesis and protection, treatment of neurodegenerative diseases such as Alzheimer's disease and neuropathy, cartilage protection, bone differentiation promotion, treatment of knee osteoarthritis, renal fibrosis alleviation, improvement of diabetic endothelial cell dysfunction, prevention of diabetic nephropathy cell apoptosis, islet β cell protection, blood sugar reduction, diabetes prevention and has the potential of treating metabolic diseases.

### BRIEF DESCRIPTION OF THE DRAWINGS

FIG. 1 is a graph showing a comparison of the insulin content of a conditioned medium CnSF-CM and a conditioned medium SpSF-CM in insulin content analysis.

FIG. 2 is a graph showing an analysis of the extracellular vesicle, wherein A is a panel showing the particle size distribution of the extracellular vesicle in the conditioned medium CnSF-CM and the conditioned medium SpSF-CM, B is a panel showing statistical comparison of the extracellular vesicle concentrations of the conditioned medium CnSF-CM and the conditioned medium SpSF-CM, and C is a panel showing a statistical comparison of the particle size of the extracellular vesicle of the conditioned medium CnSF-CM and the conditioned medium SpSF-CM.

FIG. 3 is a diagram showing the analysis of a cell wound healing assay, wherein A is a picture of the wound healing experiments of the conditioned medium CnSF-CM and the conditioned medium SpSF-CM, and B is a quantitative statistical comparison diagram of the cell wound healing assays of the conditioned medium CnSF-CM and the conditioned medium SpSF-CM.

### DESCRIPTION OF THE EMBODIMENTS

In order to make the objective, technical features and advantages of the present invention more understandable to those skilled in the relevant art and to implement the present invention, the technical features and implementations of the present invention are specifically explained with reference to the accompanying drawings, and better embodiments are listed for further explanation. The referred drawings hereafter are schematic representations related to the characteristics of the present invention, and are not and need not be completely drawn according to the actual situation.

Herein, all technical and scientific terms used herein have the same meaning as commonly understood by one of ordinary skills in the art to which the present invention pertains. Furthermore, unless clearly contradicted otherwise by the context, as used herein, singular terms shall include pluralities, and plural terms shall include the singular.

Although the numerical ranges and parameters used for defining a broader scope of the present invention are approximate values, the relevant numerical values in the specific embodiments have been presented as accurately as possible. Any numerical value, however, inherently contains inevitable standard deviations resulting from individual testing methods. As used herein, “about” generally means that the actual numerical value is within plus or minus 10%, 5%, 1% or 0.5% of a specified numerical value or range. Alternatively, the term “about” means that the actual numerical value falls within an acceptable standard error from the mean, as determined by one of ordinary skills in the art to which the invention pertains. Except for the examples, or unless otherwise specified, it is to be understood that all ranges, quantities, numerical values and percentages used herein (e.g., for describing the amount of materials, time length, temperature, operating conditions, quantity ratio and the like) are all modified by “about”. Therefore, unless otherwise stated to the contrary, the numerical parameters disclosed in this specification and the appended claims are all approximate values and may be changed as required. At a minimum, these numerical parameters should be understood as the indicated significant digits and the numerical values obtained by applying a general carry method.

In order to make the description of the disclosure more detailed and complete, the following provides an illustrative description of the implementation mode and specific embodiments of the present invention; however, this is not the only form of implementing or using the specific embodiments of the present invention. The embodiments cover the features of multiple specific embodiments as well as the method steps for constructing and operating these specific embodiments and the order thereof. However, other specific embodiments may also be used for achieving the same or equivalent functions and step sequences.

The pharmaceutical composition of the present invention is prepared by the following steps:
(a) a step of preparing a pharmaceutically effective mother liquor: a starting stem cell is implanted into a basal medium, and cultured the starting stem cell under conditions of an ambient temperature of 35.5-39.5° C. and a CO₂ concentration of 5% for more than at least 2 days, and then the starting stem cell is removed to obtain a pharmaceutically effective mother liquor suitable for wound repair; and
(b) a step of preparing a pharmaceutical composition: treating the pharmaceutical effective mother solution by a purification and refining means to obtain a pharmaceutical composition suitable for treating wound repair.

The starting stem cell is a human mesenchymal stem cell, or a cultured stem cell obtained by culturing the human mesenchymal stem cell. The source of the human mesenchymal stem cell used in the present invention is not particularly limited. For example, the human mesenchymal stem cell can be selected from any of an adipose-derived stem cell, a bone marrow stem cell, a peripheral blood stem cell, and an umbilical cord blood stem cell, and preferably selected from the adipose-derived stem cell or the bone marrow stem cell.

As mentioned above, the cultured stem cell is obtained by implanting the human mesenchymal stem cell into an auxiliary medium and culturing them statically for at least 24 hours so that the cells adhere to a cell plate. The auxiliary medium is a keratinocyte serum-free medium containing 5-20% of fetal bovine serum, 1-100 mM of N-acetyl-L-cysteine, and 0.05-50 mM of L-ascorbic acid 2-phosphate.

Furthermore, in step a, the basal medium is a serum-free medium containing a growth factor or growth hormone, a redox substance, an energy metabolism-related ingredient, and other nutrient sources.

The serum-free medium is selected from a serum-free DMEM/F12 medium, a RPMI 1640 serum-free medium, or a EMEM serum-free medium, and preferably the serum-free DMEM/F12 medium.

The growth factor or growth hormone of the basal medium is at least one selected from the group consisting of activin-A, corticosterone, exendin-4, a hepatocyte growth factor (HGF), pentagastrin, progesterone, retinol, and triiodol-1-thyronine. The redox substance is at least one selected from the group consisting of catalase, glutathione, and superoxidase dismutase. The energy metabolism-related ingredient is at least one selected from the group consisting of D(+)-galactose, ethanolamine, L-carnitine, linoleic acid, linolenic acid, lipoic acid, nicotinamide, putrescine, and sodium selenite. The other nutrient sources are at least one selected from the group consisting of albumin, D, L-alpha-tocopherol, D, L-alpha-tocopherol acetate, holo transferrin, and retinol acetate.

In the basal medium, the content of the activin-A is generally between 0.1-100 pM, preferably between 0.1-50 pM, more preferably between 0.5-50 pM, and most preferably between 1-10 pM.

In the basal medium, the content of corticosterone is generally between 0.1-200 nM, preferably between 0.1-100 nM, more preferably between 0.5-100 nM, and most preferably between 1-100 nM.

In the basal medium, the content of exendin-4 is generally between 1-100 nM, preferably between 1-50 nM, more preferably between 5-50 nM, and most preferably between 5-20 nM.

In the basal medium, the content of the hepatocyte growth factor (HGF) is generally between 40-500 fM, preferably between 40-250 fM, more preferably between 80-250 fM, and most preferably between 80-120 fM.

In the basal medium, the content of pentagastrin is generally between 1-100 nM, preferably between 1-50 nM, more preferably between 5-50 nM, and most preferably between 5-20 nM.

In the basal medium, the content of progesterone is generally between 1-300 nM, preferably between 1-200 nM, more preferably between 10-200 nM, and most preferably between 10-100 nM.

In the basal medium, the content of retinol is generally between 50-900 nM, preferably between 50-700 nM, more preferably between 100-700 nM, and most preferably between 100-500 nM.

In the basal medium, the content of triiodol-1-thyronine is generally between 0.05-40 nM, preferably between 0.05-20 nM, more preferably between 0.1-20 nM, and most preferably between 0.1-10 nM.

In the basal medium, the content of catalase is generally between 0.5-200 nM, preferably between 0.5-100 nM, more preferably between 1-100 nM, and most preferably between 1-50 nM.

In the basal medium, the content of glutathione is generally between 0.5-40 uM, preferably between 0.5-20 uM, more preferably between 1-20 uM, and most preferably between 1-10 uM.

In the basal medium, the content of superoxide dismutase is generally between 25-600 nM, preferably between 25-400 nM, more preferably between 50-400 nM, and most preferably between 50-200 nM.

In the basal medium, the content of D(+)-galactose is generally between 0.5-600 uM, preferably between 0.5-400 uM, more preferably between 1-400 uM, and most preferably between 1-200 uM.

In the basal medium, the content of ethanolamine is generally between 0.5-400 uM, preferably between 0.5-200 uM, more preferably between 1-200 uM, and most preferably between 1-100 uM.

In the basal medium, the content of L-carnitine is generally between 0.5-400 uM, preferably between 0.5-200 uM, more preferably between 1-200 uM, and most preferably between 1-100 uM.

In the basal medium, the content of linoleic acid is generally between 0.05-40 uM, preferably between 0.05-20 uM, more preferably between 0.1-20 uM, and most preferably between 0.1-10 uM.

In the basal medium, the content of linolenic acid is generally between 0.05-40 uM, preferably between 0.05-20 uM, more preferably between 0.1-20 uM, and most preferably between 0.1-10 uM.

In the basal medium, the content of lipoic acid is generally between 5-900 nM, preferably between 5-700 nM, more preferably between 10-700 nM, and most preferably between 10-500 nM.

In the basal medium, the content of nicotinamide is generally between 1-100 mM, preferably between 1-50 mM, more preferably between 5-50 mM, and most preferably between 5-30 mM.

In the basal medium, the content of putrescine is generally between 0.5-800 uM, preferably between 0.5-500 uM, more preferably between 1-500 uM, and most preferably between 1-300 uM.

In the basal medium, the content of sodium selenite is generally between 0.5-400 nM, preferably between 0.5-200 nM, more preferably between 1-200 nM, and most preferably between 1-100 nM.

In the basal medium, the content of albumin is generally between 0.5-400 uM, preferably between 0.5-200 uM, more preferably between 1-200 uM, and most preferably between 1-100 uM.

In the basal medium, the content of D,L-alpha-tocopherol is generally between 0.05-40 uM, preferably between 0.05-20 uM, more preferably between 0.1-20 uM, and most preferably between 0.1-10 uM.

In the basal medium, the content of D,L-alpha-tocopherol acetate is generally between 0.05-40 uM, preferably between 0.05-20 uM, more preferably between 0.1-20 uM, and most preferably between 0.1-10 uM.

In the basal medium, the content of holo transferrin is generally between 0.5-600 nM, preferably between 0.5-400 nM, more preferably between 1-400 nM, and most preferably between 1-200 nM.

In the basal medium, the content of retinol acetate is generally between 1-900 nM, preferably between 1-700 nM, more preferably between 10-700 nM, and most preferably between 10-500 nM.

Preferably, the basal medium is a serum-free DMEM/F12 medium including activin-A, albumin, catalase, corticosterone, D(+)-galactose, D, L-alpha-tocopherol, D,L-alpha-tocopherol acetate, ethanolamine, exendin-4, glutathione, a hepatocyte growth factor (HGF), holo transferrin, L-carnitine, linoleic acid, linolenic acid, lipoic acid, nicotinamide, pentagastrin, progesterone, putrescine, retinol acetate, retinol, sodium selenite, superoxidase dismutase, and triiodol-1-thyronine.

Also, in step a, the starting stem cell can be removed by means of centrifugation and/or filtration to obtain the pharmaceutically effective mother liquor.

As mentioned above, the purification and refining means in step (b) are mainly to remove cell debris, impurities, bacteria and large vesicles from the pharmaceutically effective mother liquor. The purification and refining means include: centrifuging the pharmaceutically effective mother liquor at an acceleration of 300 g for 5 minutes, then removing a precipitate, and then centrifuging at an acceleration of 4,000 g for 20 minutes, and removing a precipitate again. Moreover, after completing the aforementioned centrifugation procedure, the purification and refining means may further include: filtering the pharmaceutically effective mother liquor after centrifuging by a filter with a pore size of 0.22 μm.

The pharmaceutical composition obtained by the aforementioned steps at least includes an extracellular vesicle, insulin, and a plurality of growth factors; wherein the concentration of the extracellular vesicle in the pharmaceutical composition is (57.53±3.79)×10⁸ particles/mL; the concentration of the insulin in the pharmaceutical composition is 34,870.83±7,335.13 mU/L; these growth factors are ANGPTL4, HGF, G-CSF, PDGF-AA, VEGF-A, IL-18BPa, COMP, MMP-1, MMP-2, MMP-3, MMP-7, MMP-9, MMP-10, and MMP-12; and the extracellular vesicle contains a CLU protein, a TIMP1 protein and a YWHAB protein.

The concentration of ANGPTL4 in the pharmaceutical composition is 24.87±1.21 ng/ml. The concentration of HGF in the pharmaceutical composition is 0.77±0.01 ng/ml. The concentration of G-CSF in the pharmaceutical composition is 932.00±33.14 pg/ml. The concentration of PDGF-AA in the pharmaceutical composition is 80.80±3.59 pg/ml. The concentration of VEGF-A in the pharmaceutical composition is 577.88±10.28 pg/ml. The concentration of IL-18BPa in the pharmaceutical composition is 6.57±1.15 pg/ml. The concentration of COMP in the pharmaceutical composition is 4,686.50±143.54 pg/ml. The concentration of MMP-1 in the pharmaceutical composition is 21,412.50±1,576.14 pg/ml. The concentration of MMP-2 in the pharmaceutical composition is 45,930.50±809.64 pg/ml. The concentration of MMP-3 in the pharmaceutical composition is 383,733.00±17,703.13 pg/ml. The concentration of MMP-7 in the pharmaceutical composition is 293.97±0.00 μg/ml. The concentration of MMP-9 in the pharmaceutical composition is 13.74±1.20 pg/ml. The concentration of MMP-10 in the pharmaceutical composition is 78.10±3.95 pg/ml. The concentration of MMP-12 in the pharmaceutical composition is 150.20±7.21 pg/ml.

In addition to being used for wound repair-related treatments such as repair of diabetic wounds, repair of pressure sores, corneal repair, repair of xerophthalmia, repair of various difficult wounds or wounds that have not healed for a long time, skin repair or maintenance in medical cosmetology, the pharmaceutical composition can also be used for angiogenesis, immune diseases, heart protection, treatment of cardiovascular diseases, heart damage repair, treatment of ischemic heart diseases, neurogenesis and protection, treatment of neurodegenerative diseases such as Alzheimer's disease and neuropathy, cartilage protection, bone differentiation promotion, treatment of knee osteoarthritis, renal fibrosis alleviation, improvement of diabetic endothelial cell dysfunction, prevention of diabetic nephropathy cell apoptosis, islet β cell protection, blood sugar reduction, diabetes prevention and has the potential of treating metabolic diseases.

Next, the present invention is described hereafter with reference to specific examples.

### Example 1 and Comparative Example 1

Mesenchymal stem cells (human adipose mesenchymal stem cells were employed in the present invention) were cultured in a keratinocyte serum-free medium containing 5-20% fetal bovine serum, 1-100 mM of N-acetyl-L-cysteine, and 0.05-50 mM of L-ascorbic acid 2-phosphate. During the culture process, the temperature was controlled at 36.5-38.5° C., and the cells were cultured in a cell culture incubator containing 5% carbon dioxide to amplify the cell number.

After the mesenchymal stem cells are expanded to the required number for the experiment, they are inoculated into a cell plate at a cell density of 6,000-15,000 cells/cm² and added into an auxiliary medium for culture. The auxiliary medium is a keratinocyte serum-free medium containing 5-20% fetal bovine serum, 1-100 mM of N-acetyl-L-cysteine, and 0.05-50 mM of L-ascorbic acid 2-phosphate. After culture for 1 day, the cells were attached to the cell plate, then the auxiliary medium was removed, and the cells were washed twice with dPBS.

Then, the mesenchymal stem cells were divided into two groups, Example 1 and Comparative Example 1. The mesenchymal stem cells of Comparative Example 1 were cultured in a control serum-free medium (CnSF medium) for 7 days, while the mesenchymal stem cells of Example 1 were cultured in a basal medium for 7 days. The serum-free medium of the control group was a keratinocyte serum-free medium containing 1-100 mM of N-acetyl-L-cysteine and 0.05-50 mM of L-ascorbic acid 2-phosphate. The composition of the basal medium was shown in Table 1 below.

TABLE 1
Basal medium

| | | Concentration | |
|---|---|---|---|
| Ingredient | | range | |
| Serum-free DMEM/F12 medium | | — | |
| Growth factor or | Activin-A | 1-10 | pM |
| growth hormone | Corticosterone | 1-100 | nM |
| | Exendin-4 | 5-20 | nM |
| | HGF | 80-120 | fM |
| | Pentagastrin | 5-20 | nM |
| | Progesterone | 10-100 | nM |
| | Retinol | 100-500 | nM |
| | Triiodol-l-thyronine | 0.1-10 | nM |
| Redox substance | Catalase | 1-50 | nM |
| | Glutathione | 1-10 | uM |
| | Superoxidase dismutase | 50-200 | nM |
| Energy metabolism- | D(+)-galactose | 1-200 | uM |
| related ingredient | Ethanolamine | 1-100 | uM |
| | L-Carnitine | 1-100 | uM |
| | Linoleic acid | 0.1-10 | uM |
| | Linolenic acid | 0.1-10 | uM |
| | Lipoic acid | 10-500 | nM |
| | Nicotinamide | 5~30 | mM |
| | Putrescine | 1-300 | uM |
| | Sodium Selenite | 1-100 | nM |
| Other nutrient sources | Albumin | 1-100 | uM |
| | D,L-alpha-Tocopherol | 0.1-10 | uM |
| | D,L-alpha-Tocopherol acetate | 0.1-10 | uM |
| | Holo transferrin | 1-200 | nM |
| | Retinol acetate | 10-500 | nM |

After 7 days, the supernatants of the two groups were removed and transferred to new tubes. They were centrifuged at an acceleration of 300 g for 5 minutes, then removed and transferred to new tubes and centrifuged at an acceleration of 4,000 g for 20 minutes. The supernatants were then removed and filtered with a 0.22 μm filter (Merck Millipore, Billerica, MA, USA). Cell debris, impurities, bacteria and large vesicles were removed through the aforementioned steps to obtain the conditioned medium CnSF-CM of Comparative Example 1 and the conditioned medium SpSF-CM of Example 1 (i.e., the pharmaceutical composition of the present invention). The aforementioned conditioned medium CnSF-CM and the conditioned medium SpSF-CM were then subjected to cytokine and growth factor content detection, insulin content detection, extracellular vesicle analysis, extracellular vesicle purification and concentration and content identification, and cell wound healing assay, respectively.

### Cytokine and Growth Factor Content Detection

In order to know the content of cytokines and growth factors in the conditioned medium CnSF-CM and the conditioned medium SpSF-CM, a detection instrument MILLIPLEX® MAP MULIPLEX DETECTION (Merck Milliplex, instrument model: Luminex Magpix analyzer) was used for detecting the content of cytokines and growth factors in a physiological saline solution (negative control group), the conditioned medium CnSF-CM and the conditioned medium SpSF-CM, and the results were recorded in Table 2.

TABLE 2

| | Group | | | | | | |
|---|---|---|---|---|---|---|---|
| | Physiological saline | | | | | | |
| | solution | | | | | | The difference |
| | (Negative control | | | | | | multiple |
| | group) | | CnSF-CM | | SpSF-CM | | between SpSF- |
| | | Standard | | Standard | | Standard | CM and CnSF- |
| Factor (unit) | Average | Deviation | Average | Deviation | Average | Deviation | CM |
| ANGPTL4 | 0.79 | 0.00 | 0.79 | 0.00 | 24.87 | 1.21 | 31.47 |
| (ng/ml) | | | | | | | |
| HGF (ng/ml) | 0.01 | 0.00 | 0.01 | 0.00 | 0.77 | 0.01 | 76.50 |
| G-CSF (pg/ml) | 2.54 | 0.53 | 2.16 | 0.00 | 932.00 | 33.14 | 431.48 |
| PDGF-AA | 7.19 | 0.20 | 4.60 | 0.37 | 80.80 | 3.59 | 17.57 |
| (pg/ml) | | | | | | | |
| VEGF-A | 1.60 | 0.08 | 1.35 | 0.04 | 577.88 | 10.28 | 429.65 |
| (pg/ml) | | | | | | | |
| IL-18BPa | 1.42 | 0.00 | 1.42 | 0.00 | 6.57 | 1.15 | 4.62 |
| (pg/ml) | | | | | | | |
| COMP (pg/ml) | 3.64 | 0.00 | 3.64 | 0.00 | 4686.50 | 143.54 | 1287.50 |
| MMP-1 (pg/ml) | 2.90 | 0.00 | 3.17 | 0.37 | 21412.50 | 1576.14 | 6765.40 |
| MMP-2 (pg/ml) | 83.56 | 0.00 | 5.62 | 0.00 | 45930.50 | 809.64 | 8172.69 |
| MMP-3 (pg/ml) | 60.73 | 0.00 | 51.46 | 0.00 | 383733.00 | 17703.13 | 7456.92 |
| MMP-7 (pg/ml) | 188.80 | 0.00 | 188.80 | 0.00 | 293.97 | 0.00 | 1.56 |
| MMP-9 (pg/ml) | 3.28 | 0.44 | 2.97 | 0.00 | 13.74 | 1.20 | 4.62 |
| MMP-10 | 6.52 | 0.00 | 6.52 | 0.00 | 78.10 | 3.95 | 11.98 |
| (pg/ml) | | | | | | | |
| MMP-12 | 1.00 | 0.00 | 0.82 | 0.00 | 150.20 | 7.21 | 183.17 |
| (pg/ml) | | | | | | | |

The experimental results showed that the content of cytokines and growth factors in the conditioned medium SpSF-CM was higher than that in the physiological saline solution and the conditioned medium CnSF-CM. Further multiple calculation was conducted, and it showed that the conditioned medium SpSF-CM contained 31.47 times more ANGPTL4, 76.50 times more HGF, 431.48 times more G-CSF, 17.57 times more PDGF-AA, 429.65 times more VEGF-A, 4.62 times more IL-18BPa, 1,287.50 times more COMP, 6,765.40 times more MMP-1, 8172.69 times more MMP-2, 7456.92 times more MMP-3, 1.56 times more MMP-7, 4.62 times more MMP-9, 11.98 times more MMP-10, and 183.17 times more MMP-12 compared with the conditioned medium CnSF-CM, indicating that the cytokines and growth factors rich in the conditioned medium SpSF-CM obtained through Example 1 must be produced by co-culturing with the mesenchymal stem cells in the basal medium, while in Comparative Example 1, co-culturing with the mesenchymal stem cells in the control serum-free medium could not produce a large amount of specific cytokines and growth factors.

An ANGPTL4 protein was involved in fat metabolism regulation and therefore had the potential to control weight and promote angiogenesis and wound healing, had anti-inflammatory ability and promoted heart injury repair. A HGF protein was an important growth factor in vivo, participating in the development and repair of body organs, promoting cell growth, inhibiting fibrosis, inhibiting cell apoptosis, combating against inflammation and promoting angiogenesis. A G-CSF protein could promote growth, survival, and differentiation of hemocytes, promote nerve cell generation and reduce cell apoptosis. A PDGF-AA protein could promote angiogenesis, promote wound healing and promote osteogenic differentiation. A VEGF-A protein promoted angiogenesis and increased vascular permeability for the treatment of neurodegenerative diseases, neuropathy, and ischemic heart diseases. A IL-18Bpa protein was involved in immune regulation. A COMP protein had a protective effect on cartilage and the vascular system. A COMP supplement was a potential therapy for osteoarthritis and cardiovascular diseases. MMP series proteins were involved in wound repair, angiogenesis, and immune regulation.

### Insulin Content Detection and Extracellular Vesicle Analysis

The conditioned medium CnSF-CM and the conditioned medium SpSF-CM were detected for insulin content (model: Chemiluminescence, Atellica IM, SIEMENS), and the results were recorded in Table 3. Moreover, the conditioned medium CnSF-CM and the conditioned medium SpSF-CM were subjected to extracellular vesicle analysis (model: NTA, NanoSight NS300; Malvern Panalytical, Malvern, UK), and the results were recorded in Table 3.

TABLE 3

| | | Conditioned medium | |
|---|---|---|---|
| | | CnSF-CM | SpSF-CM |
| Number of mesenchymal stem | | 1.167 × 10⁶ | 1.167 × 10⁶ |
| cells as used | | | |
| Culturing days | | 7 days | 7 days |
| Insulin concentration (mU/L) | | 1.7 ± 0.69 | 34870.83 ± 7335.13 |
| Extracellular | Quantity (10⁸ | 8.61 ± 0.28 | 57.53 ± 3.79 |
| vesicle | particles/ml) | | |
| | Particle size (nm) | 112.27 ± 2.08 | 122.63 ± 1.01 |

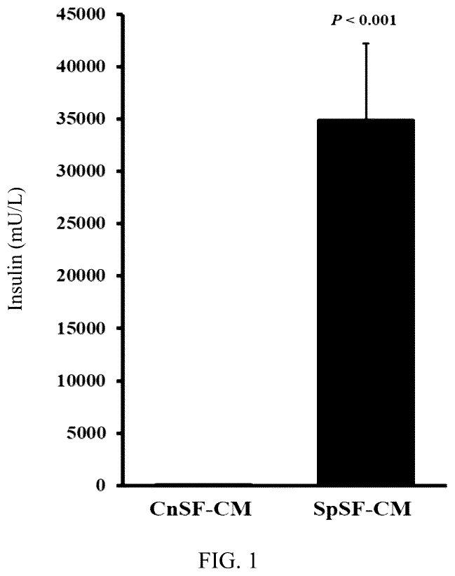

As shown in the results of Table 3 and FIG. 1, the insulin content of the conditioned medium SpSF-CM was 34,870.83±7,335.13 mU/L, and the insulin content of the conditioned medium CnSF-CM was 1.7±0.69 mU/L. The insulin concentration of the conditioned medium SpSF-CM was 20,512 times that of the conditioned medium CnSF-CM.

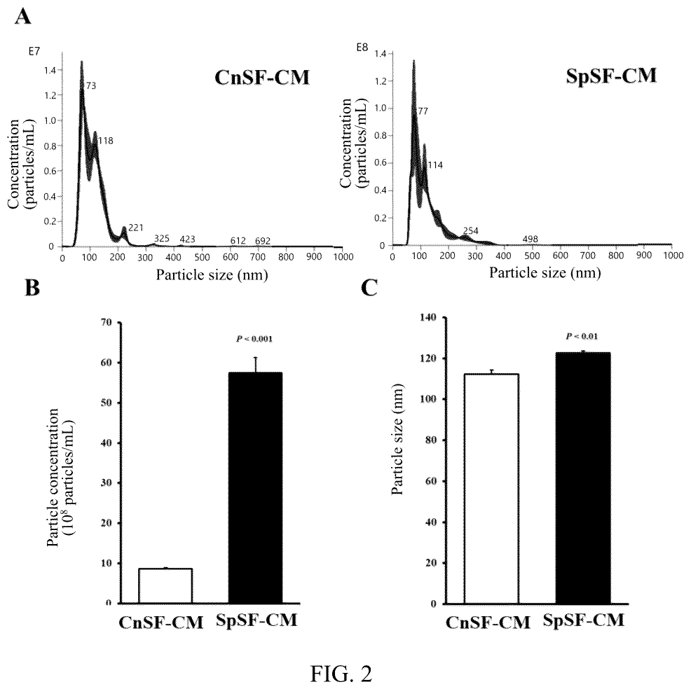

Furthermore, as shown in the results of Table 3 and FIG. 2, the number of the extracellular vesicles in the conditioned medium SpSF-CM was 57.53±3.79×10⁸ particles/ml, and the number of the extracellular vesicles in the conditioned medium CnSF-CM was 8.61±0.28×10⁸ particles/ml. The number of the extracellular vesicles in the conditioned medium SpSF-CM was 6.7 times that of the conditioned medium CnSF-CM. The particle size of the extracellular vesicles in the conditioned medium SpSF-CM was 122.63±1.01 nm, the particle size of the extracellular vesicles in the conditioned medium CnSF-CM was 112.27±2.08 nm, and the particle size of the extracellular vesicles in the conditioned medium SpSF-CM was slightly larger than that in the conditioned medium CnSF-CM.

### Purification, Concentration and Content Identification of Extracellular Vesicle

In order to understand the extracellular vesicle contents of the conditioned medium CnSF-CM and the conditioned medium SpSF-CM, the extracellular vesicles were further purified and concentrated by centrifugation at an acceleration of 4,000 g using a 100 kDa Amicon Ultra-15 Centrifugal Filter Device (Millipore). After the centrifugation, the upper layer of the separation tube was separated to obtain a concentrated solution of the extracellular vesicles, which was then subjected to extracellular vesicle analysis (instrument model: NTA, NanoSight NS300; Malvern Panalytical, Malvern, UK) to obtain the number of the extracellular vesicles. Then, a quantitative number of 2×10⁸ extracellular vesicles was taken from each group for extracellular vesicle content identification. The experimental steps were illustrated as follows and the analysis results were recorded in Table 4.

Gel assisted digestion—the protein solution was mixed with a SDS-PAGE sample buffer and incubated in an environment at a temperature of 95° C. for 10 minutes. The protein was then loaded and analyzed by 10% SDS-PAGE (1 cm) stained with Coomassie blue. The gel was cut and destained, then reduced with 10 mM dithiothreitol (DTT, Merck) in an environment at a temperature of 60° C. for 45 minutes, and then subjected to cysteine-blocking with 55 mM iodoacetamide (IAM, Sigma) in an environment at a temperature of 25° C. for 30 minutes. The specimen was digested with sequencing-grade modified porcine trypsin (Promega) at 37° C. for 16 hours. The peptide was then extracted from the gel, dried by vacuum centrifugation, and reconstituted with 0.1% formic acid, and then equal volumes of the samples were analyzed by LC-MS/MS.

LC-MS/MS Analysis—the digested peptide was diluted in a HPLC buffer A (0.1% formic acid) and loaded onto a reverse phase column (Zorbax 300SB-C18, 0.3×5 mm; Agilent Technologies). Then, the desalted peptide was separated in a self-made column (Waters BEH 1.7 μm, 100 μm I.D. 10 cm with a 15 μm tip) by using the multi-step gradient of a HPLC buffer B (99.9% acetonitrile/0.1% formic acid) at a flow rate of 0.3 μl/min for 70 minutes. The LC device was coupled to a 2D linear ion trap mass spectrometer (Orbitrap Elite ETD; Thermo Fisher) operated using Xcalibur 2.2 software (Thermo Fisher). Full scan MS was conducted in an Orbitrap with a range of 400 to 2,000 Da, a resolution of 120,000, and m/z 400. The protonated dodecamethylcyclohexasiloxane ion with an ion signal of m/z 536.165365 was used as the lock mass for internal calibration. After 20 data-related MS/MS scanning events, the 20 most abundant precursor ions in the preview MS scanning were subjected to MS scanning once. The m/z value selected for MS/MS was dynamically excluded for 40 seconds, the relative mass window was 15 ppm, the electrospray voltage was set to 2.0 kV, and the capillary temperature was set to 200° C. MS and MS/MS automatic gain control was set to 1,000 ms (full scan) and 200 ms (MS/MS), or 3×10⁶ ions (full scan) and 3,000 ions (MS/MS) to obtain maximum accumulation time or ions, respectively.

Protein Identification and Label-free Quantitation-data analysis was conducted using Proteome Discoverer software (version 2.3, Thermo Fisher Scientific). A MS/MS spectrogram was searched in the Swissprot database using a Mascot search engine (Matrix Science, London, UK; version 2.5). For peptide identification, an error of 10 ppm was allowed for the intact peptide mass, an error of 0.5 Da was allowed for the CID fragment ion, and two missed cutting sites resulting from trypsin digestion were allowed: oxidized methionine and acetyl (at the N-terminal of the protein) as variable modifications; and carbamidomethyl (cysteine) as a static modification. Peptide spectrogram matches (PSMs) were then filtered according to the peptide identification with high confidence and ranked first in a Mascot search engine to ensure an overall false discovery rate of less than 0.01. Proteins with single peptide hits were removed. Relative quantification of proteins was based on the sum of peptide peak areas as detected by a Minora algorithm. 2-fold change was used as a cutoff to select differential proteins.

The results were recorded in Table 4.

TABLE 4

| | | Expression multiple (compared to CnSF-CM | |
|---|---|---|---|
| | | group) | |
| Conditioned medium | | CnSF-CM | SpSF-CM |
| Protein name | CLU | 1 | 3.13 |
| | TIMP1 | 1 | 2.73 |
| | YWHAB | 1 | 10.63 |

The CLU protein had the potential of nerve protection, heart protection, pain regulation, immunity regulation, treatment of ocular diseases such as xerophthalmia, treatment of neurodegenerative diseases such as Alzheimer's disease, reduction of renal fibrosis, improvement of diabetic endothelial cell dysfunction, prevention of diabetic nephropathy cell apoptosis, and protection of pancreatic β cells. The TIMP1 protein was involved in the potential of wound repair, regeneration, immune regulation, angiogenesis, and diabetes prevention. The YW HAB protein had the potential of lowering blood sugar and treating metabolic diseases.

From the results in Table 4 above, it could be seen that when a certain amount (2×10 8 particles) of the extracellular vesicles were taken for content analysis, it was found that the conditioned medium SpSF-CM increased the amount of the CLU protein that helped repair of the corneal epithelial cells by 3.13 times, the amount of the TIMP1 protein that promoted wound healing by 2.73 times, and the amount of the YWHAB protein by 10.63 times compared to the conditioned medium CnSF-CM. The results indicated that the extracellular vesicles in the conditioned medium SpSF-CM contained higher contents of the CLU, TIMP1, and YWHAB proteins, which was speculated to enhance the wound repair effect.

### Cell Wound Healing Assay

In order to test the effects of the conditioned medium CnSF-CM and the conditioned medium SpSF-CM on wound repair, a cell wound healing assay was conducted. First, skin fibroblasts were cultured with DMEM/F12+10% FBS and the cells were expanded for experimental use. Then, the skin fibroblasts were seeded in a 6-well plate at a cell number of 2×10⁵ with 2×10⁵ cells per well, and cultured for one day. The medium was removed the next day, and the cells were washed once with dPBS. Then, the cells were added with a serum-free DMEM/F12 medium and cultured for 24 h to allow the remaining nutrients to be completely metabolized. The next day, 200 ul of Tips was utilized to conduct cell scraping to simulate wound generation (drawing a line), then the medium was removed, and the cells were washed twice with dPBS to remove scraped and floating cells.

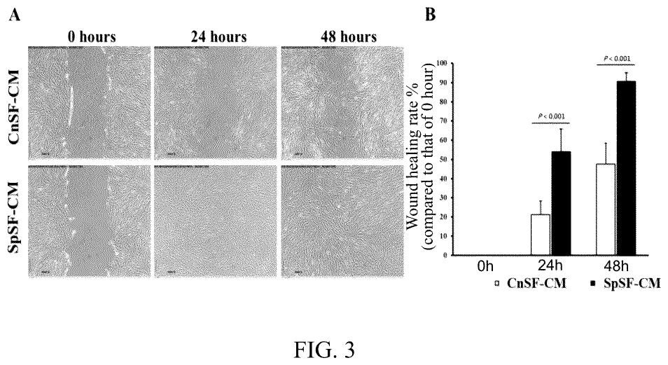

Each 3 ml of the conditioned medium CnSF-CM and the conditioned medium SpSF-CM was then added, and the cells in the 6-well culture plate dish were photographed and observed using an inverted fluorescence microscope (model: OLYMPUS IX71-1LL100) at 0, 24, and 48 hours respectively. Thereafter, Image J software was used for calculating and statistically analyzing the wound healing area. The results were recorded in Table 5 and FIG. 3.

TABLE 5

| Conditioned | | | Wound healing rate % (compared to that at 0 hour) | |
|---|---|---|---|---|
| medium | | | CnSF-CM | SpSF-CM |
| Time | 0 | h | 0 | 0 |
| | 24 | h | 21.30 ± 7.02 | 47.55 ± 10.96 |
| | 48 | h | 54.09 ± 11.73 | 90.73 ± 4.32 |

The experimental results showed that the wound healing degrees were 47.55% and 21.30% respectively after the skin fibroblasts were cultured in the conditioned medium SpSF-CM and the conditioned medium CnSF-CM respectively for 24 hours, and were 90.73% and 54.09% after culture for 48 hours. No matter whether it was 24 hours or 48 hours, when compared under the same culture time, the repair ability of the conditioned medium SpSF-CM was about twice that of the CnSF-CM, showing the excellent wound healing potential of the conditioned medium SpSF-CM. The combination of the extracellular vesicles, insulin and the growth factors contained in it can be used as a pharmaceutical composition for wound repair.

In summary, the disclosure of the present invention has been explained by the aforementioned embodiments, but the present invention is not limited to these embodiments. Various changes and modifications can be made by those skilled in the art to which the present invention pertains without departing from the spirit and scope of the present invention. For example, the various technical contents illustrated in the aforementioned embodiments may be combined or modified to form a new implementation, and such implementations shall of course be regarded as one of the disclosure of the present invention. Therefore, the claimed scope of in this application also includes the claims described hereafter and the scope defined thereby.

What is claimed is:

1. A method for preparing a pharmaceutical composition for treating wound repair, which comprises:
(a) a step of preparing a pharmaceutically effective mother liquor: implanting a starting stem cell into a basal medium, and culturing the starting stem cell under conditions of an ambient temperature of 35.5-39.5° C. and a CO₂ concentration of 5% for more than at least 2 days, and then removing the starting stem cell to obtain a pharmaceutically effective mother liquor suitable for wound repair; and
(b) a step of preparing a pharmaceutical composition: treating the pharmaceutical effective mother solution by a purification and refining means to obtain a pharmaceutical composition suitable for treating wound repair; wherein
the starting stem cell is a human mesenchymal stem cell, or a cultured stem cell obtained by culturing the human mesenchymal stem cell;
the pharmaceutically effective mother liquor at least contains an extracellular vesicle, insulin, and a plurality of growth factors as pharmaceutically effective ingredients;
the basal medium is a serum-free medium composed of a growth factor or growth hormone, a redox substance, an energy metabolism-related ingredient, and other nutrient sources.

2. The method for preparing a pharmaceutical composition for treating wound repair according to claim 1, wherein the growth factor or growth hormone of the basal medium is at least one selected from the group consisting of activin-A, corticosterone, exendin-4, a hepatocyte growth factor (HGF), pentagastrin, progesterone, retinol, and triiodol-1-thyronine.

3. The method for preparing a pharmaceutical composition for treating wound repair according to claim 1, wherein the redox substance of the basal medium is at least one selected from the group consisting of catalase, glutathione, and superoxidase dismutase.

4. The method for preparing a pharmaceutical composition for treating wound repair according to claim 1, wherein the energy metabolism-related ingredient of the basal medium is at least one selected from the group consisting of D(+)-galactose, ethanolamine, L-carnitine, linoleic acid, linolenic acid, lipoic acid, nicotinamide, putrescine, and sodium selenite.

5. The method for preparing a pharmaceutical composition for treating wound repair according to claim 1, wherein the other nutrient sources of the basal medium are at least one selected from the group consisting of albumin, D, L-alpha-Tocopherol, D, L-alpha-Tocopherol acetate, holo transferrin, and retinol acetate.

6. The method for preparing a pharmaceutical composition for treating wound repair according to claim 1, wherein the cultured stem cell is obtained by implanting the human mesenchymal stem cell into an auxiliary medium and culturing for at least 24 hours; and
the auxiliary medium is a keratinocyte serum-free medium containing fetal bovine serum, N-acetyl-L-cysteine, and ascorbyl phosphate.

7. The method for preparing a pharmaceutical composition for treating wound repair according to claim 1, wherein the cultured stem cell is obtained by implanting the human mesenchymal stem cell at a cell density of 6,000 to 15,000 cells/cm² into an auxiliary medium for at least 24 hours, and the auxiliary medium is a keratinocyte serum-free medium containing fetal bovine serum, N-acetyl-L-cysteine, and ascorbyl phosphate.

8. The method for preparing a pharmaceutical composition for treating wound repair according to claim 1, wherein the purification and refining means comprises: centrifuging the pharmaceutically effective mother liquor at an acceleration of 300 g for 5 minutes, then removing a precipitate, and then centrifuging at an acceleration of 4,000 g for 20 minutes, and removing a precipitate again.

9. The method for preparing a pharmaceutical composition for treating wound repair according to claim 8, wherein the purification and refining means further comprises: filtering the pharmaceutically effective mother liquor after centrifugation by a filter with a pore size of 0.22 μm.

10. A pharmaceutical composition for treating wound repair, which is prepared by the preparation method of any one of claim 1, wherein the pharmaceutical composition at least comprises an extracellular vesicle, insulin, and a plurality of growth factors, wherein
a concentration of the extracellular vesicle in the pharmaceutical composition is (57.53±3.79)×10⁸ particles/mL;
a concentration of the insulin in the pharmaceutical composition is 34,870.83±7,335.13 mU/L;
these growth factors are ANGPTL4, HGF, G-CSF, PDGF-AA, VEGF-A, IL-18BPa, COMP, MMP-1, MMP-2, MMP-3, MMP-7, MMP-9, MMP-10, and MMP-12; and
the extracellular vesicle contains a CLU protein, a TIMP1 protein and a YWHAB protein.
